(19) United States
(12) Patent Application Publication
(10) Pub. No.: US 2007/0053204 A1
(43) Pub. Date: Mar. 8, 2007

(54) Confocal microendoscope comprising optical fibres with a tapering diameter

(75) Inventors: Ingo Krohne, Aachen (DE); Andre Ehrhardt, Tuttlingen (DE); Klaus M. Irion, Liptingen (DE); Herbert Stepp, Planegg (DE)

Correspondence Address: PURDUE LAW OFFICES;2735 N. HOLLAND-SYLVANIA ROAD, SUITE B-2, TOLDEO, OH, 43615, US

(73) Assignees: FRAUNHOFER-GESELLSCHAFT ZUR FORDERUNG DER ANGEWAND, Munchen (DE); KARL STORZ GMBH & CO. KG, Tuttlingen (DE)

(21) Appl. No.: 10/573,141

(22) PCT Filed: Sep. 10, 2004
(86) PCT No.: PCT/DE04/02035

§ 371 Date: Mar. 22, 2006

(30) Foreign Application Priority Data
Sep. 22, 2003 (DE) 103 44 169.7

(51) Int. Cl. A61B 3/00; A61B 1/06; G03B 29/00; G03B 15/02; G02B 6/06; F21V 5/00

(52) U.S. Cl. 362/574; 600/160

(57) ABSTRACT

The invention relates to a confocal microendoscope, in which the diameter of optical fibres at the proximal end (8) of an optical fibre bundle (9) is greater than at the distal end (15). This permits the efficiency of coupling the light of a light source to be increased, without reducing the resolution of the microendoscope. In addition, the proximal ends (8) of the optical fibres (10) are arranged in a grid, for example, by means of a fibre receiving unit (11) that holds the individual optical fibres. A microlens unit (13) can also be provided, said unit allocating a microlens (14) to each optical fibre end.

The invention relates to a confocal endomicroscope comprising a light source, a fiber optic bundle having a proximal end and a distal end, and a micromirror unit for injecting the light from the light source into the proximal end of the fiber optic bundle.

An endomicroscope of the type mentioned in the introduction is disclosed, for example, by the publication “New Concept for the Development of a Confocal Endomicroscope” by I. Krohne et al., 36^{th} Annual Congress of the DGBMT, 2002, volume 47, pages 206 to 208. Confocal microscopy is based on imaging a point light source through suitable optics onto the object to be measured. In an endomicroscope, the light is delivered to the object to be measured via an optical fiber or a multiplicity of optical fibers in a bundle. The light is reflected back by the object through the optical fiber or fibers via a beam splitter onto the detector element. If the object to be measured lies in the focal point of the distal end of the optical fiber, then the reflected light will be imaged onto the detector with its full intensity. This is not the case when the object to be measured lies outside the focal point. In this case, a pinhole before the detector element holds back some of the reflected light. The axial height information is therefore encoded in an intensity distribution typical for confocal microscopy. When a fiber optic bundle is used, the object to be measured may be scanned by injecting the light from the light source successively into the proximal ends of the individual optical fibers of the bundle. To this end, it is necessary to know the relationship between the position of the distal ends of the individual optical fibers and their proximal ends.

By using light point patterns, it is also possible to inject a plurality of light points simultaneously into different optical fibers, in order to shorten the measuring time. A micromirror unit, with the aid of which the individual fibers are successively illuminated for the scanning, is used for controlled injection of the light into the proximal ends of the individual optical fibers.

It is difficult to achieve a high efficiency of the light injection into the proximal ends of the optical fibers. For instance, it is substantially necessary to ensure that the light intended for a particular optical fiber is not injected into neighboring optical fibers, since otherwise this would significantly limit the resolution. Light photons which reach the cladding material of the optical fibers or enter the gaps between the optical fibers likewise reduce the efficiency, since they will be back-scattered and thereby possibly lower the contrast, or even are scattered into neighboring optical fibers.

U.S. Pat. No. 4,938,205 describes an endoscope for imaging from regions of the body interior and for treating these regions by exposure to energetic radiation. To this end, it has one or more channels which may in turn contain individual optical fibers or fiber optic bundles. Several versions with different arrangements of optical channels and associated light sources and/or sensors are described. An exemplary design relates to a scanner camera, for example having a laser as the light source, with which the region of interest is scanned. For therapy, a high-energy laser beam may alternatively or additionally be injected into one or more fibers, depending on the intended application. It is mentioned that in conventional endoscopes this high-energy laser radiation often leads to damage of the fibers at the proximal end. The problem here is not the optical fibers themselves, since they merely conduct most of the energy, but rather the holding and cladding material which surrounds them and will be damaged by excessive heating or heat shock. In order to resolve this problem, the optical fibers of an optical channel may be widened at the proximal end so that, for example, they have the shape of an elongated conic frustum, the diameter of the fibers at the proximal end being substantially greater than that at the distal end. It is thereby possible to achieve a higher heat capacity and better cooling possibilities. Values of 10:1 and 4:1 are mentioned for the ratio of the diameters of the fibers at the proximal and distal ends. The fibers are respectively fixed at both ends of the fiber bundle, in order to ensure coherence. The use of such tapering fibers for purposes other than for introducing high-energy radiation for treatment is not envisaged nor implied, and in particular no imaging application is presented. In the described embodiments and examples, the photosensors are for the most part fitted to the distal end of the endoscope.

Methods for the production of one- and two-dimensional arrays of optical fibers for parallel rapid data transmission are described in the dissertation “Parallele optische Verbindungsnetzwerke mit zweidimensionalen Koppelelementen” [parallel optical connection networks with two-dimensional coupling elements] by Uwe Danzer. Various construction technologies are proposed for one- and two-dimensional fiber matrices, which make it possible to arrange the ends of optical fibers in a defined grid with high accuracy. Interaction with microlens rows or arrays is also presented, and a parallel point-to-point connection of 256 fibers constructed thereby is described. The fields of application are preferably data transmission and the construction of communication networks.

It is therefore an object of the invention to provide a confocal endomicroscope of the type mentioned in the introduction, in which the efficiency of the light input into the proximal end of the individual optical fibers can be increased significantly compared with the prior art.

This object is achieved by an endomicroscope of the type mentioned in the introduction in that the diameter of the optical fibers of the fiber optic bundle is greater at the proximal end than at the distal end.

A minimal diameter of the optical fibers is desirable at the distal end of the fiber optic bundle, in order to be able to achieve a high resolution. The larger diameter of the optical fibers at their proximal end at the same time ensures the possibility of being able to inject a sufficient light intensity into the individual optical fibers. Perturbing side effects, such as illumination of the optical fiber cladding or the gaps between the optical fibers, can be reduced or even entirely avoided.

The endomicroscope according to the invention may also be designed so that the optical fibers taper essentially conically from the proximal end to the distal end. This provides uniform, monotonic tapering which means that only very minor perturbations of the light conduction are to be expected.

It may furthermore be advantageous to design the endomicroscope according to the invention so that the ratio of the diameters of the optical fibers at the proximal end to the diameters of the optical fibers at the distal end is at most 3.

Besides a relatively short fiber optic bundle with diameters of the optical fibers tapering toward the distal end, the endomicroscope according to the invention may comprise a further fiber optic bundle of constant diameter following on from the proximal end of the first fiber optic bundle.

The endomicroscope according to the invention may furthermore be designed such that the optical fibers are arranged in a fixed grid at the proximal end of the fiber optic bundle.

Fixing the proximal optical fiber ends in a grid is an important measure in order to be able to address the individual optical fiber ends in a controlled and accurate way for the light injection. It is then expedient to select the grid such that no two optical fiber ends are directly adjacent, in order to be able to substantially avoid injection into a plurality of fibers. To this end, the optical fibers should be separated at their proximal end.

The arrangement of the optical fiber ends in the grid may, for example, be hexagonal or square. Compared with a square arrangement, a hexagonal arrangement has the advantage of a higher packing density in the fiber bundle, and therefore better resolution. A hexagonal structure is furthermore particularly favorable in respect of manufacturing the fiber bundle.

As an alternative to a two-dimensional grid, a linear arrangement of the optical fiber ends could also be conceivable. In order to be able to transmit two-dimensional image information, a plurality of linear bundles should then be stacked on one another. Compared with a two-dimensional grid, this has the disadvantage of an additional error source due to the stacking.

In order to fix the fiber ends in their position, it may be expedient to design the endomicroscope such that a fiber holding unit with openings to hold the proximal fiber ends is provided for the arrangement in a grid.

The fiber holding unit may be manufactured micromechanically, which allows a very high positioning accuracy of the individual fibers relative to one another. Knowing the exact positions of the individual fibers at the proximal end simplifies calibration of the endomicroscope, so that it is even possible to use incoherent fiber optic bundles. Using incoherent fiber optic bundles can reduce the costs of the overall system. Machining methods or even, for example, silicon technology may be envisaged for micromechanical manufacture of the fiber holding unit, in order to achieve the desired position accuracy.

Lastly, the endomicroscope according to the invention may be designed such that a microlens unit is arranged in the radiation direction before the proximal end of the fiber optic bundle, so that the light is focused by the individual microlenses onto the proximal end of the illuminated optical fibers. The injection efficiency can be further improved in this way. With a hexagonal arrangement, for an equal packing density, it is possible to select a larger linear spacing of the microlenses in the microlens unit compared with a square arrangement, so that it is possible to achieve a correspondingly better addressability and therefore a better injection efficiency.

A preferred embodiment of the endomicroscope according to the invention will be explained below with reference to figures, in which:

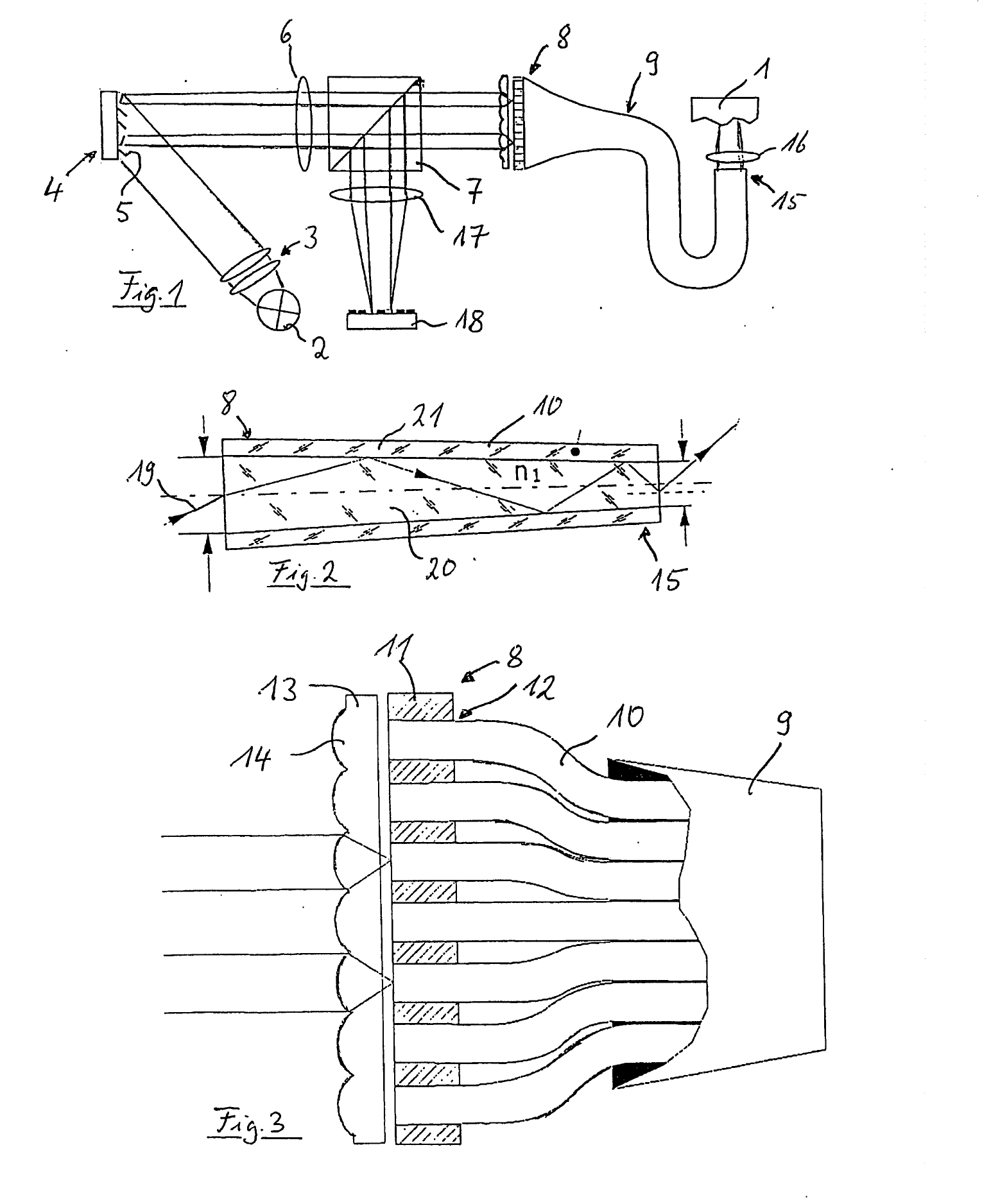

FIG. 1 schematically shows the structure of an endomicroscope in use,

FIG. 2 schematically shows a lateral cross section through an individual optical fiber, and

FIG. 3 schematically shows details of the proximal end of the fiber optic bundle.

FIG. 1 shows schematically and in a very simplified way the structure of an endomicroscope for studying an object 1 to be measured. The light from a light source 2 is directed via source optics 3 onto a micromirror unit 4. The micromirror unit 4 consists of hundreds of individual micromirrors, each of which can be controlled individually for tilting movements. For the simplified representation, only a few of the micromirrors 5 are represented schematically and greatly enlarged in FIG. 1. The light is injected into the proximal end 8 of a fiber optic bundle 9 via mirror optics 6 and a beam splitter 7. An enlarged representation of the proximal end 8 of the fiber optic bundle 9 is partially represented in FIG. 3. Accordingly, the individual optical fibers 10 are separated at the proximal end 8 and arranged in a grid. A fiber holding unit 11, which comprises openings 12 intended for the individual optical fibers 10 and matched to the diameters of the optical fibers 10 at the proximal end 8, is provided for fixing the ends of the optical fibers 10 at the proximal end 8. The optical fibers 10 may, for example, be arranged in a hexagonal pattern or in a square pattern in the fiber holding unit 11. Before the proximal end 8 of the optical fibers 10, a microlens unit 13 is provided so that a microlens 14 is arranged in front of each proximal end 8 of every optical fiber 10.

There are two different procedures for injecting the light into the proximal end 8 of the fiber optic bundle 9: on the one hand, a single light beam may be injected successively into the proximal ends 8 of the individual optical fibers 10; on the other hand, it is also possible to expose a plurality of optical fiber ends to a respective light beam simultaneously, in order to reduce measuring times.

At the distal end 15 of the fiber optic bundle 9, the light emerges and travels via output optics 16 onto the object 1 to be measured. From the surface of the object 1 to be measured, or from structures inside the object 1 to be measured, the emerging light beams are reflected back into the fiber optic bundle. The reflected light then enters essentially the same optical fiber 10 from which it previously emerged.

The reflected light travels via the fiber optic bundle 9, the microlens unit 13, the beam splitter 7 and via detector optics 17 onto a detector unit 18, for example a CCD camera. Each pixel of the detector unit 18 may be allocated to a proximal end of a particular optical fiber 10. If a coherent fiber optic bundle 9 is used, the allocation of each pixel to a distal optical fiber end derives directly from this. If an incoherent fiber optic bundle 9 is used, it is first necessary to calibrate it. This is done, for example, by injecting predetermined light/dark patterns into the proximal end 8 and evaluating the light/dark distribution established at the distal end 15.

Efficient injection of the light into the individual optical fibers 10 is already possible owing to the arrangement of the optical fibers 10 in a predetermined grid at the proximal end 8, and owing to the use of the microlens unit 13. This efficiency is increased even further, without reducing the resolution of the endomicroscope, by the fact that the diameters of the optical fiber ends 10 at their proximal end 8 is greater than at their distal end 15, for example by a factor of about 2.5.

FIG. 2 shows, in cross section and in a foreshortened form, an optical fiber 10 whose diameter monotonically tapers conically from the proximal end 8 to the distal end 15. The course of an injected light ray 19 when it enters, inside an optical fiber core 20, and when it emerges at the distal end 15 is represented. The light ray 19 is reflected inside the optical fiber 10 from the optical fiber cladding 21.

### LIST OF REFERENCES

1 object to be measured
2 light source
3 source optics
4 micromirror unit
5 micromirror
6 mirror optics
7 beam splitter
8 proximal end
9 fiber optic bundle
10 optical fiber
11 fiber holding unit
12 openings
13 microlens unit
14 microlens
15 distal end
16 output
17 detector optics
18 detector unit
19 light ray
20 optical fiber core
21 optical fiber cladding

1-6. (canceled)

7. A confocal endomicroscope comprising a light source (2), a fiber optic bundle (9) having a proximal end (8) and a distal end (15), and a micromirror unit (4) for injecting the light from the light source (2) into the proximal end (8) of the fiber optic bundle (9), characterized in that the diameter of the optical fibers (10) of the fiber optic bundle (9) is greater at the proximal end (8) than at the distal end (15) and that the optical fibers (10) are arranged in a fixed grid at the proximal end (8) of the fiber optic bundle (9).

8. The confocal endomicroscope as claimed in claim 7, characterized in that the optical fibers (10) taper essentially conically from the proximal end (8) to the distal end (15).

9. The confocal endomicroscope as claimed in claim 7 or 8, characterized in that the ratio of the diameters of the optical fibers (10) at the proximal end (8) to the diameters of the optical fibers (10) at the distal end (15) is equal to or less than 3.

10. The confocal endomicroscope as claimed in one of claims 7 or 8, characterized in that a fiber holding unit (11) with openings to hold the proximal fiber ends is provided for the arrangement in a grid.

11. The confocal endomicroscope as claimed in claim 9, characterized in that a fiber holding unit (11) with openings to hold the proximal fiber ends is provided for the arrangement in a grid.

12. The confocal endomicroscope as claimed in one of claims 7 or 8, characterized in that a microlens unit (13) is arranged in the radiation direction before the proximal end (8) of the fiber optic bundle (9), so that the light is focused by the individual microlenses (14) onto the proximal end (8) of the illuminated optical fibers (10).

13. The confocal endomicroscope as claimed in claims 9, characterized in that a microlens unit (13) is arranged in the radiation direction before the proximal end (8) of the fiber optic bundle (9), so that the light is focused by the individual microlenses ( 14) onto the proximal end (8) of the illuminated optical fibers (10).

14. The confocal endomicroscope as claimed in claim 10, characterized in that a microlens unit (13) is arranged in the radiation direction before the proximal end (8) of the fiber optic bundle (9), so that the light is focused by the individual microlenses (14) onto the proximal end (8) of the illuminated optical fibers (10).

15. The confocal endomicroscope as claimed in claim 11, characterized in that a microlens unit (13) is arranged in the radiation direction before the proximal end (8) of the fiber optic bundle (9), so that the light is focused by the individual microlenses (14) onto the proximal end (8) of the illuminated optical fibers (10).
